(19) United States
(12) Patent Application Publication
(10) Pub. No.: US 2025/0371957 A1
(43) Pub. Date: Dec. 4, 2025

(54) Multi-Functional Device Aiding Carers of Small Children

(71) Applicant: Baby Patent Ltd., Ramat Gan (IL)

(72) Inventor: Oren SHRAK, Ramat Gan (IL)

(21) Appl. No.: 19/225,184

(22) Filed: Jun. 2, 2025

Related U.S. Application Data
(60) Provisional application No. 63/655,257, filed on Jun. 3, 2024.

(51) Int. Cl. G08B 21/02; A61B 5/01

(52) U.S. Cl. CPC G08B 21/0211; A61B 5/01; G08B 21/0208; A61B 2503/04

(57) ABSTRACT

A multi-functional device for small children, the device typically comprising a thermometer for evaluating a small child's body temperature. Typically coupled thereto, there may be a set of functional devices including a vibrator and/or speaker and/or microphone and/or night-light to enable a carer or parent to efficiently and effectively minister to the small child upon her or his waking and/or when night-feeding and/or when she or he cries.

### FIELD OF THIS DISCLOSURE

The present invention relates generally to multi-functional devices, and more particularly to multi-functional child-care devices.

### BACKGROUND

Multi-functional devices are extremely useful and convenient, such as the renowned Swiss Army Knife, a variant of which was patented on 12 Jun. 1897; a multifunctional pocket tool, with a knife or knife-pliers plus removable screwing attachments, was much later issued the following patent: patents.google.com/patent/U.S. Pat. No. 5,809,600A/en.

Multi-functional devices for babies include, for example, the Hatch Rest Baby Sound Machine, Night Light, Sleep Trainer, Time-to-Rise Alarm Clock, White Noise Soother which uses Bluetooth or Wi-Fi Technology, and includes a sound machine producing soothing sounds like white noise, stories, ocean, wind, birds, heartbeat, rain, lullabies, a night-light to illuminate the crib and changing table, Time-to-Rise functionality which uses color and sound to let a child know whether to rise or stay in bed, and a control app to adjust settings with a smartphone, or control volume and the night-light by touch with buttons on the device. The product is intended “for Nursery, Toddler, & Kids Bedroom”.

Hatch Rest Go is a portable sound machine for babies and kids with a baby sleep soother which clips onto a baby stroller.

KOPUO is a baby monitor with a digital camera, display video with night vision, video baby monitor with camera and audio, temperature monitor (“When the room temperature reaches the set temperature, the screen will sound to remind you”), and Secure Privacy Wireless Color LCD Tech.

Vtechphones.com distributes a video baby monitor with pan and tilt and night-light. “Pan, tilt and zoom the camera from the parent unit. Watch as your little one moves about. The camera pans up to 270 degrees side-to-side and tilts as many as 25 degrees up and down . . . the temperature indicator on the parent unit lets you know about when to adjust the thermostat.”

Another multi-functional device for babies is a bathroom device described in U.S. Pat. No. 7,999,198 to Shrak et al.

Some nursery multi-functional devices for babies include a sleep-trainer. For example, luvion.com's “Bear sleep trainer with night-light” can be set to a desired time. The Luvion light goes on half an hour before the set time (yellow) indicating almost time to get up and gets get brighter as wake up time approaches, at which set time, the light becomes green, indicating that the child, if awake, can get out of bed.

Vibrating pads for nurseries are known. For example, the Munchkin Lulla-Vibe Vibrating Mattress Pad “easily slips under a crib mattress or bassinet and creates gentle, soothing vibrations to help lull baby to sleep”.

Grab-and-go describes an attribute, e.g., of foods, but also of other consumer products, which is considered desirable by consumers, e.g., millennials. The meaning of the term seems to be intuitive and/or loosely defined and/or elusive, and it is not apparent what technical characteristics should be provided to yield this attribute. For example, one source may define that Grab-N-Go “lets customers walk into a store, take items from the shelf, and leave without even visiting a checkout”, whereas another definition may be that “the most traditional configuration . . . when . . . Grab-N-Go” has “shelves where customers can pick out what they want and bring it to a point-of-sale”.

The disclosures of all publications and patent documents mentioned in the specification, and of the publications and patent documents cited therein directly or indirectly, are hereby incorporated by reference, other than subject matter disclaimers or disavowals. If the incorporated material is inconsistent with the express disclosure herein, the interpretation is that the express disclosure herein describes certain embodiments, whereas the incorporated material describes other embodiments. Definition/s within the incorporated material may be regarded as one possible definition for the term/s in question.

Materiality of such publications and patent documents to patentability is not conceded.

### SUMMARY

Certain embodiments seek to provide a multi-functional device for babies and small children, the device comprising a body thermometer and, typically coupled thereto, either directly or indirectly, a vibrator and/or speaker and/or microphone and/or night-light and/or baby-monitor, all typically packaged as and/or integrated or incorporated into a single unit.

Certain embodiments seek to provide a multi-functional device for babies and small children, the device comprising plural functional components or units, such as but not limited to all or any subset of: a camera (infrared e.g.) or monitor and a night light and/or a body thermometer, and/or a vibrator and/or speaker and/or microphone, all typically packaged as and/or integrated or incorporated into a single unit.

It is appreciated that in this document, functional units (e.g. vibrator and/or speaker and/or microphone and/or night-light and/or baby-monitor and/or thermometer/s), may be coupled indirectly, e.g., if functional unit A is coupled to functional unit B which is coupled to functional unit C, then functional units A, C are deemed (indirectly) coupled.

Certain embodiments seek to provide a camera with a spherical housing which facilitates use by enabling intuitive angular positioning of the camera; a base with a recess configured to conform to a portion of a sphere having the same radius as the housing, may be used to receive, support, and stabilize the sphere.

Certain embodiments of the present invention seek to provide circuitry typically comprising at least one processor in communication with at least one memory, with instructions stored in such memory executed by the processor to provide functionalities which are described herein in detail. Any functionality described herein may be firmware-implemented or processor-implemented, as appropriate.

It is appreciated that any reference herein to, or recitation of, an operation being performed is, e.g. if the operation is performed at least partly in software, intended to include both an embodiment where the operation is performed in its entirety by a server A, and also to include any type of “outsourcing” or “cloud” embodiments in which the operation, or portions thereof, is or are performed by a remote processor P (or several such), which may be deployed off-shore or “on a cloud”, and an output of the operation is then communicated to, e.g., over a suitable computer network, and used by, server A. Analogously, the remote processor P may not, itself, perform all of the operations, and, instead, the remote processor P itself may receive output/s of portion/s of the operations from yet another processor/s P′, may be deployed off-shore relative to P, or “on a cloud”, and so forth.

There is thus provided, in accordance with at least one aspect of the present invention, a multi-functional device for small children, the device typically comprising: a thermometer e.g. for evaluating a small child's body temperature; and, coupled thereto, a set of functional devices typically including a vibrator which may be used in a vibration mode option, and/or a speaker and/or microphone, and/or a night-light, thereby to enable a carer or parent to efficiently and effectively minister to the small child upon her or his waking and/or when night-feeding and/or when she or he cries.
(i) In addition to the above features, the device according to this aspect of the presently disclosed subject matter can comprise all or any subset of features (i) to (vii) listed below, in any desired combination or permutation which is technically possible:
(ii) the set of functional devices also comprises a baby-monitor.
(iii) the device also comprises a single printed circuit board, aka PCB, which controls plural devices from among the set of functional devices and the thermometer.
(iv) the single printed circuit board, aka PCB, controls all devices in the set of functional devices, and also the thermometer.
(v) The device also comprises also comprises a single user control device, e.g., sensor ring, which provides control, e.g., on-off control for said plural devices, e.g., for both of the night light and the speaker.
(vi) said thermometer comprises a non-touch or touch-free or non-contact thermometer.
(vii) the night-light provides illumination and wherein the device comprises a translucent base member which is detachable from the set of functional devices and from the thermometer coupled thereto, and which is configured to engage the night-light and to receive illumination provided thereby, thereby to enhance the illumination when the night light is engaged with the base member, by virtue of the base member's translucency.
(viii) the device also comprises a single battery which powers plural devices from among the set of functional devices and the thermometer.

According to another aspect of the presently disclosed subject matter there is provided a child-care method comprising:
providing a multi-functional device comprising:
   providing a thermometer for evaluating a small child's body temperature;
   and providing, integrally formed with the thermometer, a set of functional devices including all or any subset of a vibrator or vibration functionality, a speaker and/or microphone, and a night-light to enable a carer/parent to efficiently and effectively minister to the small child upon her or his waking and/or when night-feeding and/or when she or he cries.

This aspect of the disclosed subject matter can comprise one or more of features (i) to (vii) listed above with respect to the device, mutatis mutandis, in any desired combination or permutation which is technically possible.

Also provided, excluding signals, is a computer program comprising computer program code means for performing any of the methods shown and described herein when said program is run on at least one computer; and a computer program product, comprising a typically non-transitory computer-usable or-readable medium e.g. non-transitory computer-usable or-readable storage medium, typically tangible, having a computer readable program code embodied therein, said computer readable program code adapted to be executed to implement any or all of the methods shown and described herein. The operations in accordance with the teachings herein may be performed by at least one computer specially constructed for the desired purposes, or a general-purpose computer specially configured for the desired purpose by at least one computer program stored in a typically non-transitory computer readable storage medium. The term “non-transitory” is used herein to exclude transitory, propagating signals or waves, but to otherwise include any volatile or non-volatile computer memory technology suitable to the application.

Any suitable processor/s, display and input means may be used to process, display, e.g., on a computer screen or other computer output device, store, and accept information such as information used by or generated by any of the methods and apparatus shown and described herein; the above processor/s, display and input means including computer programs, in accordance with all or any subset of the embodiments of the present invention. Any or all functionalities of the invention shown and described herein, such as but not limited to operations within flowcharts, may be performed by any one or more of: at least one conventional personal computer processor, workstation, or other programmable device or computer or electronic computing device or processor, either general-purpose or specifically constructed, used for processing; a computer display screen and/or printer and/or speaker for displaying; machine-readable memory such as flash drives, optical disks, CDROMs, DVDs, BluRays, magnetic-optical discs or other discs; RAMs, ROMs, EPROMS, EEPROMs, magnetic or optical or other cards, for storing, and keyboard or mouse for accepting. Modules illustrated and described herein may include any one or combination or plurality of: a server, a data processor, a memory/computer storage, a communication interface (wireless (e.g. BLE) or wired (e.g. USB)), and a computer program stored in memory/computer storage.

The above devices may communicate via any conventional wired or wireless digital communication means, e.g. via a wired or cellular telephone network or a computer network such as the Internet.

The apparatus of the present invention may include, according to certain embodiments of the invention, machine readable memory containing or otherwise storing a program of instructions which, when executed by the machine, implements all or any subset of the apparatus, methods, features, and functionalities of the invention shown and described herein. Alternatively, or in addition, the apparatus of the present invention may include, according to certain embodiments of the invention, a program as above which may be written in any conventional programming language, and optionally a machine for executing the program, such as but not limited to a general purpose computer which may optionally be configured or activated in accordance with the teachings of the present invention. Any of the teachings incorporated herein may, wherever suitable, operate on signals representative of physical objects or substances.

The embodiments referred to above, and other embodiments, are described in detail in the next section.

### BRIEF DESCRIPTION OF THE DRAWINGS

Certain embodiments of the present invention are illustrated in the following drawings:

FIGS. 1 and 2 are exploded top-to-bottom and bottom-to-top views, respectively of one improved multi-functional device for babies and small children according to one embodiment; these and all other diagrams are not necessarily to scale.

FIGS. 3a-3e show various views of the device of FIGS. 1 and 2 as assembled.

FIGS. 4 and 5 are exploded top-to-bottom and bottom-to-top views, respectively of another improved multi-functional device for babies and small children according to another embodiment;

FIGS. 6a-7 show various views of the device of FIGS. 4 and 5 as assembled.

Methods and systems included in the scope of the present invention may include some (e.g. any suitable subset) or all of the functionalities or members shown in the specifically illustrated implementations by way of example, in any suitable order, e.g., as shown.

### DETAILED DESCRIPTION OF CERTAIN EMBODIMENTS

Embodiments herein provide an improved multi-functional device for babies and small children.

The device is multi-functional in the sense that multiple child-serving functionalities are provided by a single device, including a (typically no-contact) body temperature sensor.

Alternatively or in addition, the device is multi-functional in the sense that the product is optimized both for “Nursery, Toddler & Kids Bedroom” stationary use, and for grab-and-go, in the sense of being convenient to take out of the house and use when on the go (when outside and/or when visiting a location which is not the child's bedroom or home, e.g., by being portable and by clipping onto a baby stroller). In contrast, conventional families of devices e.g. as distributed by Hatch, may provide separate products for nursery use and for grab-and-go.

Alternatively, or in addition, the device is multi-functional in the sense that the product is optimized for use at multiple ages and developmental stages.

Certain embodiments include a multi-functional baby-care device including all or any subset of the following functional modules: a vibrator for calming the baby, sound-machine (speaker generating soothing sounds and/or other audio content and/or microphone to provide 2-way audio), night-light (typically chargeable and having an 8 hour (or more) battery) and thermometer (body (e.g. via ear and/or forehead) and/or room), and, optionally, also a baby-monitor which may include a camera and/or app and may provide bi-directional video and/or bi-directional audio. The vibrator may generate vibration of the device, which, in turn induces vibration of any substrate which the device is placed upon, such as the baby's mattress or crib-bars.

The term “vibrator” as used herein is intended to include any mechanical device which generates vibrations. For example, vibration may be generated by an electric motor having an unbalanced mass on its driveshaft.

Any suitable thermometer may be used, such as the Non Contact Thermometer available online, e.g., at alibaba.com/product-detail/CE-Approved-Medical-Clinical-Fever-Household_1600829910267.html?spm=a2700.galleryofferlist.p_offer.d_title.62d4549aE AyARA&s=p.

The sound-machine may emit soothing sounds, lullabies, a recording of a parent's voice, or any other suitable audio content.

The device seeks to provide an entire parent's toolkit for evaluating and coping with the baby's ongoing state, including body temperature monitoring, all incorporated into a single device. For example, if the baby is restless, the parents can easily and quickly take the infant's temperature, without disturbing the state of being asleep.

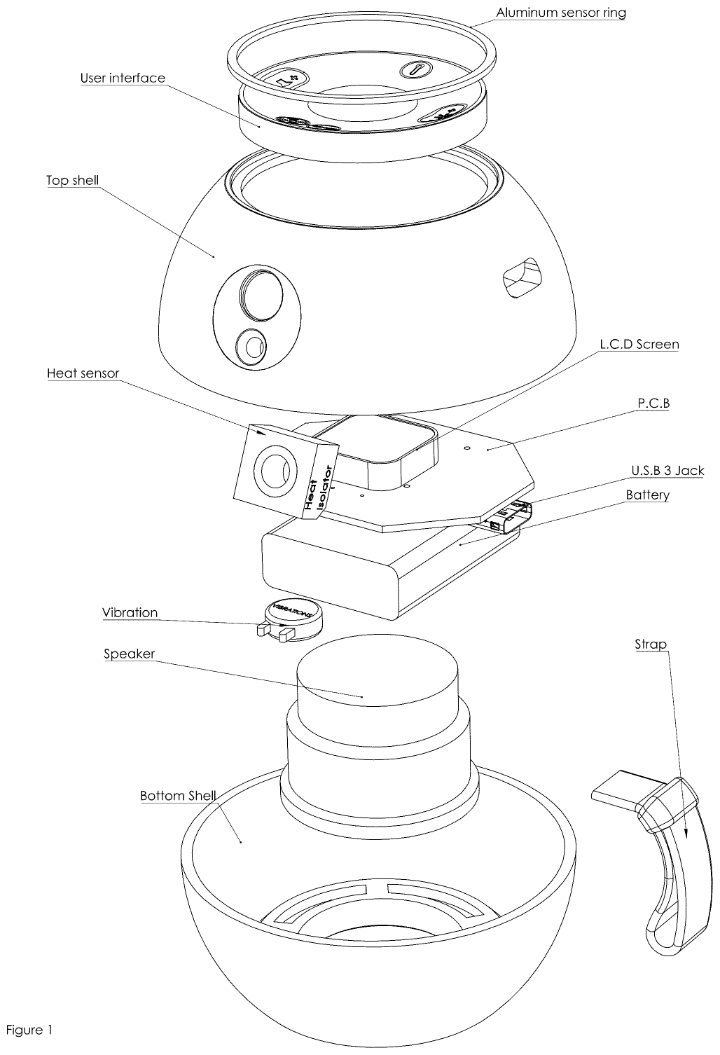

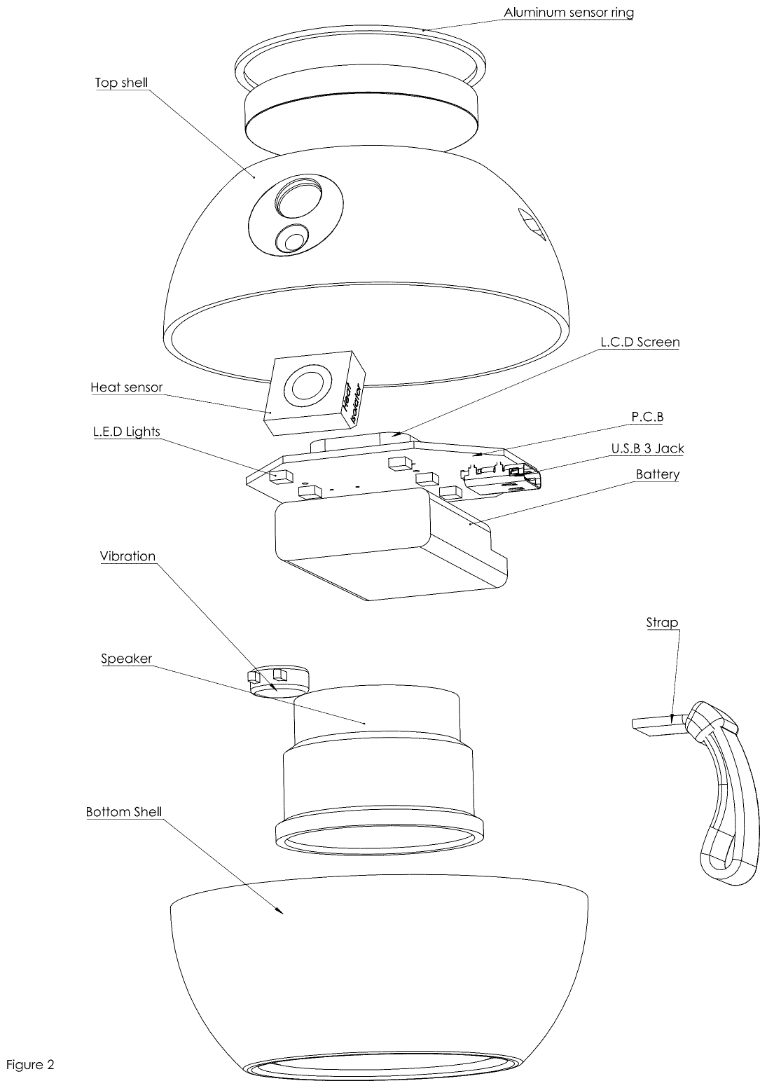

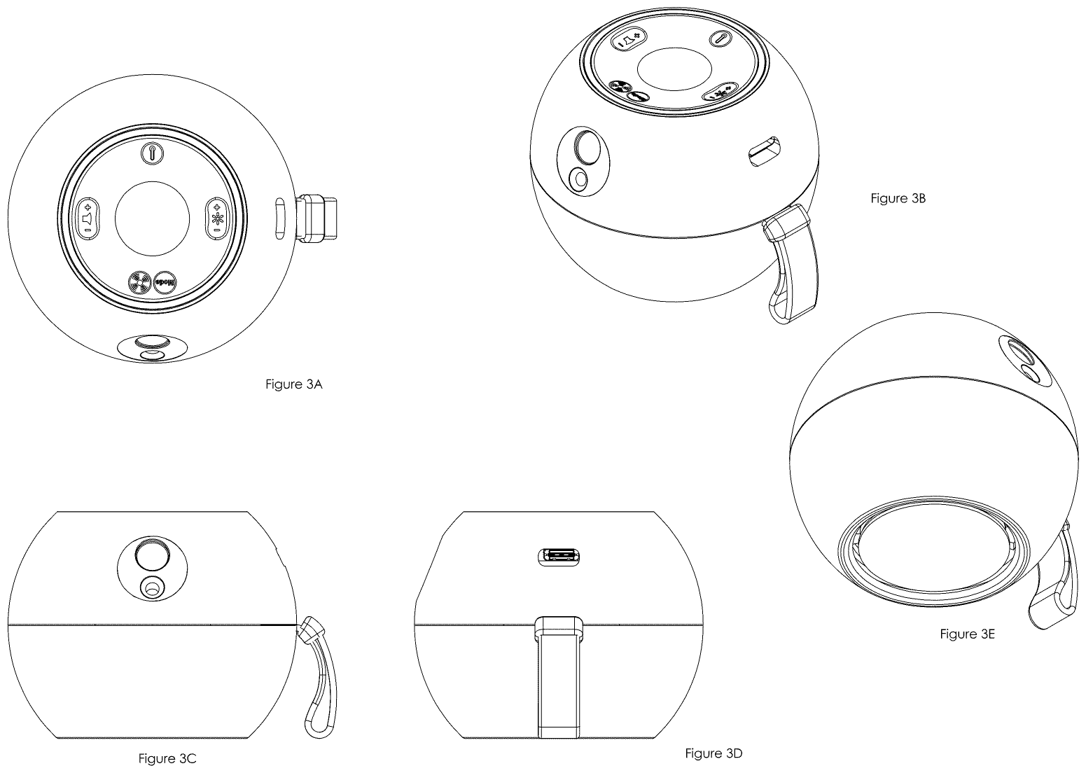

FIGS. 1 and 2 are exploded views of one improved multi-functional device for babies and small children according to certain embodiments. FIGS. 3a-3e show various views of the device of FIGS. 1-2 as assembled. The device of FIGS. 1 and 2 typically includes all or any subset of a heat sensor, vibration aka vibrator, which may be placed, say, on or under a baby's mattress to lull the baby to sleep, speaker, LCD screen to display body temperature as measured, PCB, USB jack for charging, battery, sensor ring for activation of sound and/or light, strap, and user interface, which may be deployed in any suitable manner, e.g., encased in a housing which may comprise plural, e.g., two typically generally hemispherical housing portions, aka the top and bottom shells of FIGS. 1-2.

The user interface here and in other embodiments herein, typically includes physical buttons for increasing or decreasing brightness and/or sound volume and/or for thermometer activation and/or for vibration mode activation (typically, the device vibrates only when the vibration mode/vibration functionality is turned on).

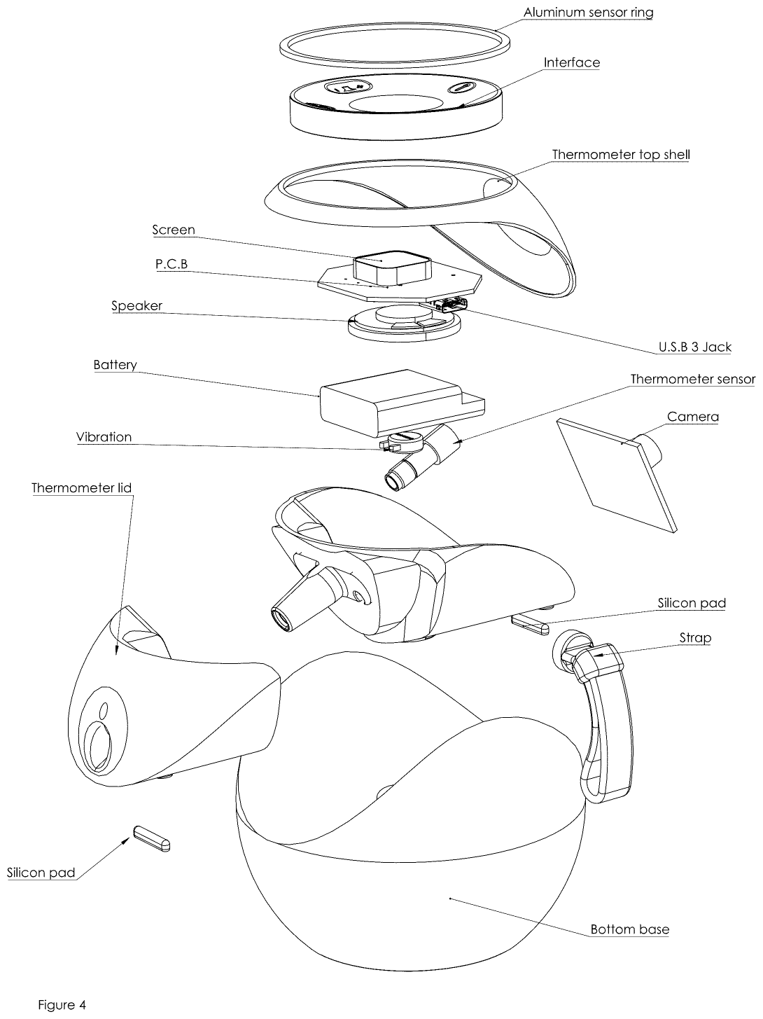

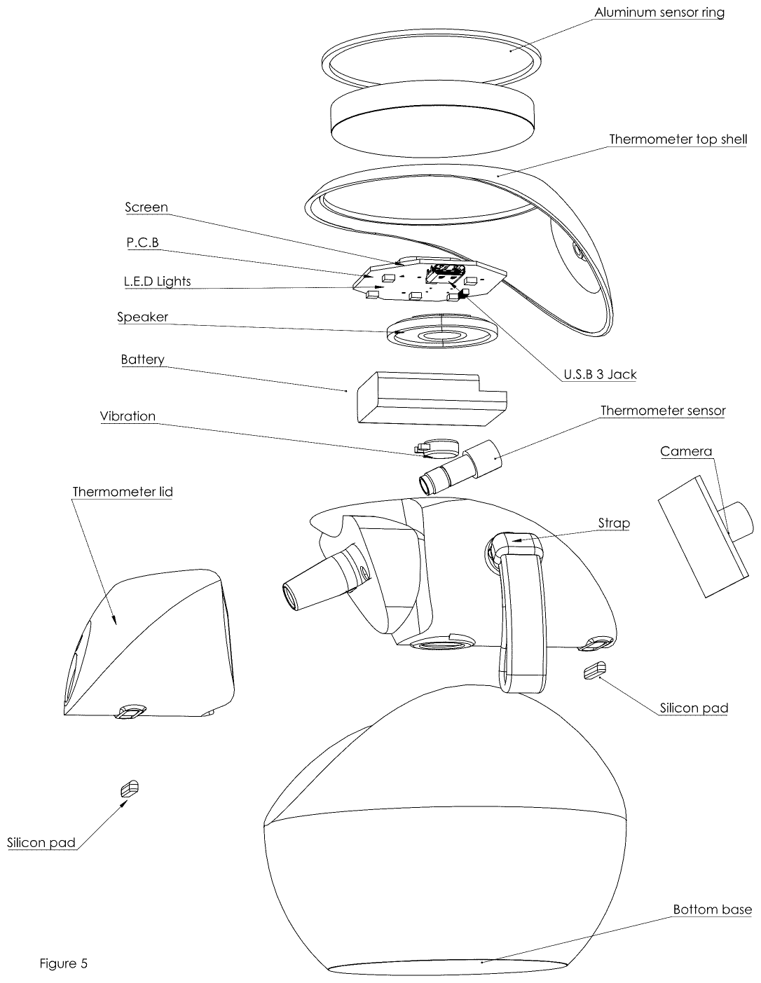

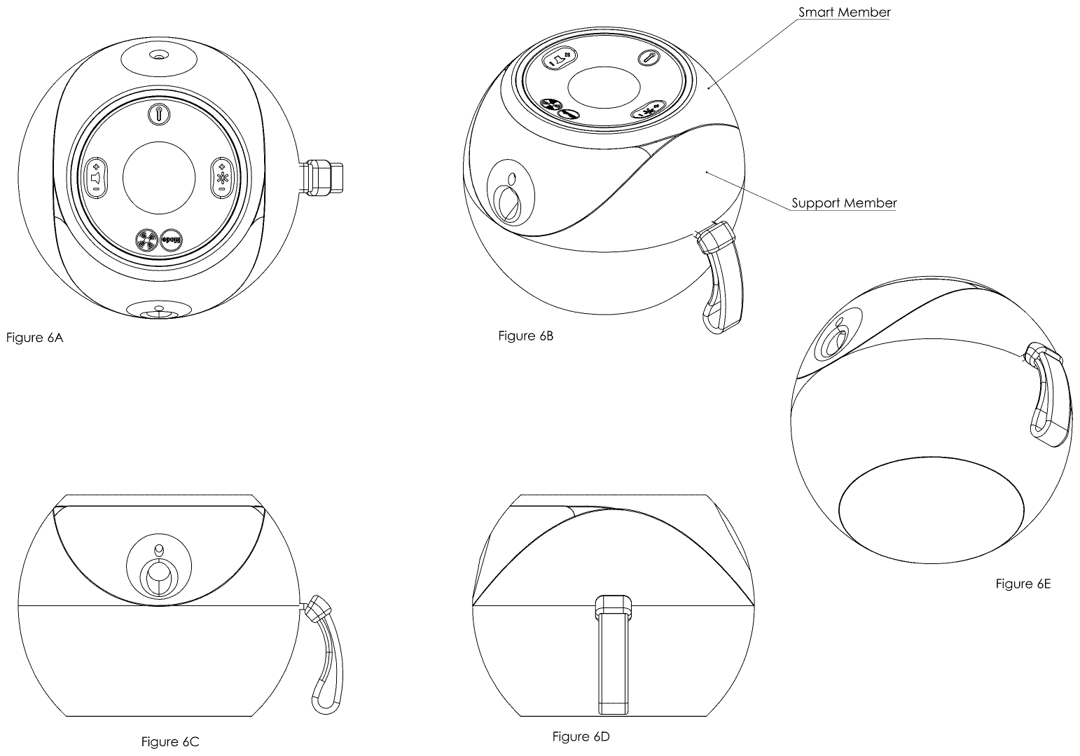

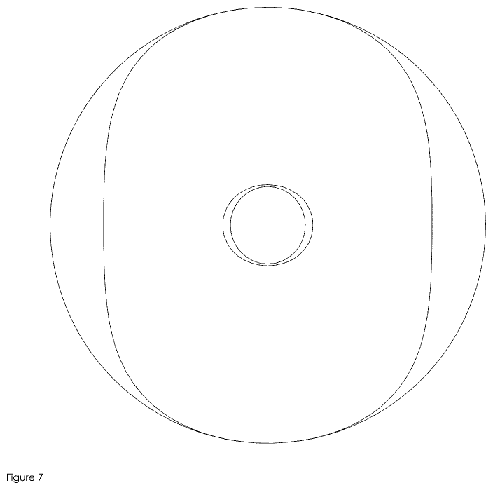

FIGS. 4 and 5 are an exploded views of another multi-functional device for babies and small children according to certain embodiments; FIGS. 6a-7 show various views of the device of FIGS. 4-5 as assembled. The system of FIGS. 4-5 may include a smart member (FIG. 6b e.g.) and support member, each of which are configured as respectively matching sphere portions such that, when joined, the two members together form a sphere. A particular advantage of this embodiment is ease of pointing the camera at the baby; rotating the sphere on its base provides easy 360 degrees of freedom in defining the field of view of the spherical camera.

Typically, the functional modules (e.g., all or any subset of the vibrator, sound-machine (microphone and/or speaker functionality), night-light, and thermometer, and baby-monitor) are all incorporated within the smart member, however the support member is typically translucent, and, therefore, when the support member is engaged with the smart member, e.g. as shown in FIG. 6b, the support member cooperates with the night-light by enhancing the illumination that the night-light provides. The support member, if configured as a portion of a sphere, may be seated on a recessed base having a recess configured as a portion of a sphere, to receive the support member. Alternatively, the support member and base may be integrally formed, e.g. of a translucent material. Thus the night-light functional module may comprise light source/s, e.g., LEDs, arranged to illuminate in the direction of the (typically translucent) support member, e.g., pointing downward or facing downward, or projecting downward (from the PCB e.g.), or tilting downward, or having a downward component. In the illustrated embodiment, the LEDs are deployed on the bottom surface of the PCB as shown in FIGS. 2 and 5 by way of example.

It is appreciated that according to certain embodiments, the same support member both enhances illumination (e.g. due to its translucency and/or light intensity amplification), and stabilizes the camera.

Baby monitoring functionality, if provided, may include a camera, and/or microphone (not shown) and/or speaker functionality, enabling the baby to be visually and aurally monitored from afar, e.g., via a cell app displaying (say, on the parent's cellphone screen), a real-time or near-real time image or video of the baby which may include a sound-track (e.g., a parent is immediately aware that the baby has begun to cry), and may even enable the parent to sooth the baby aurally from afar, e.g., by recording soothing content in real time on her or his phone, which then reaches the baby via the cell app.

The smart member typically comprises all or any subset of: a heat sensor or thermometer sensor (which here and in other embodiments herein may have ear-temperature sensing and/or forehead temperature-sensing functionality), camera, vibration, speaker, LCD screen to display, say, the child's body temperature, PCB, USB jack for charging, battery, sensor ring for activation of sound providing microphone and/or speaker functionality and/or light, strap, and a user interface, which may be deployed in any suitable manner, e.g., in association with a top shell and/or lid as shown.

Speaker/microphone functionality may for example be provided by the loudspeaker available online e.g. at alibaba.com/product-detail/2W-40mmDiameter-8-Ohm-Internal-Mini_1600213757785.html?spm=a2700.galleryofferlist.p_offer.d_title.3e2e670dyzQMu z&s=p.

The vibration functionality may utilize the following vibration motor available at alibaba.com/product-detail/Vibrator-Motor-Electric-Hot-Sale-10mm_62116238966.html?spm=a2700.galleryofferlist.p_offer.d_title.2f8260c788DiYC& s=p.

Silicon pads may be provided, e.g., as shown in FIG. 5, to enable the smart member, standalone, thus without the base, to rest securely on a surface, such as a shelf or crib mattress.

Typically, the smart member's size and durability (e.g. formed of a non-breakable material) render the device easy to transfer to and transport in a handbag, baby changing bag, diaper bag, or diaper backpack, thereby to yield grab-and-go functionality. Any adverse effect on illumination engendered by the smart member's insufficient size compared to larger night lights may be compensated, in the child's bedroom, by connecting the smart members to the translucent support member whose size may be larger by any degree than the smart member's size, thereby to achieve both grab-and-go functionality, and, when the child is in home base, very high quality illumination; it is appreciated that translucent materials soften and/or diffuse light (as opposed to clear or entirely transparent glass, which can cause glare or harsh shadows) translucent glass creates a more natural, even light which yields to illumination experienced as soft and/or gentle and/or natural and/or more even relative to lumination which does not pass through a translucent medium.

It is appreciated that the multi-functional device and/or its smart member only may be deployed in any suitable manner, and, typically, in plural selectable manners. For example, in the embodiment of FIGS. 4-5, the multi-functional device's smart member may be removably attached or coupled, e.g., clicked onto, a gripping member which grips another object, e.g., crib-bar or stroller. The gripping member may, for example, comprise a flexible elongate member, aka “snake” (which typically is formed of a material which is repeatedly bendable, yet retains its shape each time it is bent) which can be looped or wrapped (e.g., bent into a helix) around crib-bars or any other object. An example of such a “snake” is the Crib Mount Flexible Twist Adjustable Baby Monitor Stand Holder available online, e.g., at alibaba.com/product-detail/Aluminum-Alloy-Material-Easy-Installation-Universal_1600848405425.html?spm=a2700.galleryofferlist.normal_offer.d_title.5c7cca 22kqFzll.

It is appreciated that the gripping member may remain in engagement with the crib-bar (say) even while the multi-functional device's smart member is detached from the gripping member, such that clicking the multi-functional device's smart member back into engagement with the gripping member results in instant readiness of the device, without needing to re-engage the gripping member with the crib-bar or other object each time the smart-member is re-engaged (via the gripping member) with the crib-bar or other object. This means that a parent can disengage the multi-functional device's smart member from the gripping member when taking the child out for a walk, perhaps place the multi-functional device's smart member in the child's diaper bag, and then, in an instant, click the multi-functional device's smart member back into engagement with the gripping member upon returning home to put the child to sleep in her or his bed, crib, or cradle.

Alternatively, the multi-functional device's smart member may be placed on the support member, which may, in turn, rest on its base, which, in turn, may be placed on any flat surface. This enables the device to continue to serve the baby as he/she develops. For example, the device may rest on the mattress of a newborn who is, on the one hand, incapable of tampering with the device, and, is on the other hand, in possible need of the device's vibration function, for soothing. As the baby matures and becomes likely to tamper with the device, the device may be attached (via any suitable detachable connector, not shown) to the flexible elongate member, aka “snake”, which can be looped around crib-bars. Once the child is using a bed as opposed to a crib, the device may again be placed on the support member, which may, in turn, rest on its base, which, in turn, may be placed on a shelf or windowsill near the child's bed. The device or its smart member only can also be deployed on a stroller (for example, the “snake” may be looped around the bars of the stroller,) or carry-cot, or car-seat.

More generally, the device may include any suitable mechanical connector/s or fastener/s for mechanically connecting the device (or only its smart member) to any other piece of equipment, such as crib-bars, a stroller, and so forth. In addition to or instead of a mechanical connector to the “snake”, a strip of hook-and-loop fasteners, hook-and-pile fasteners, or touch fasteners, e.g., as marketed by Velcro.com, may be provided and/or any suitable strap.

Controls for all or any subset of the functional modules (e.g. on-off controls for the vibrator and/or thermometer, higher-lower volume for the sound machine, high/low brightness of night-light) may be via physical buttons on the device and/or via a touch-screen and/or via a cell app. Any suitable technology, such as Wi-Fi or Bluetooth, may be employed to provide communication between the multi-purpose device and the user's cellphone.

Where locations of various components are shown, these are not intended to be limiting. Where locations are not shown, the component is either not position-sensitive, or may be positioned as known in the art.

The multi-functional device is, according to certain embodiments, particularly useful, because unlike known devices, a parent can easily minister to a fussing, crying, or seemingly ill baby, by efficiently, and without counterproductively, worsening the situation by disturbing the baby (e.g. quickly and/or without switching on the light and/or without re-positioning the baby), check temperature, and sooth the baby using sound (e.g. lullabies) and/or vibration. This is the case for a baby in her or his crib or playpen, as well as a baby in a stroller or carriage or car-seat, or any other situation.

All or any subset of the functional components may be battery powered; all or any subset of the battery powered units may be powered by a single battery, which may, say, last for 30 hours.

Many variations are possible. Thus, for example, embodiments may provide all or any subset of the following functionalities: body and/or room temperature measurement, sound (speaker and/or microphone, light, vibration). Embodiments may or may not have a camera, may or may not have a (click) connector to a gripping member which may be wrapped around baby furniture, may or may not include a baby monitor, may or may not include a sleep trainer, and may or many not include a detachable translucent member. Thus, products according to embodiments described herein may be smaller or larger, may have more or less electronics, may generally be more or less costly, depending e.g., on whether a parent may already have purchased a separate baby-monitor (or some other functionality), may have a housing, which may or may not be single-piece, and may be associated with support or housing which may include plural members, e.g., two shells or more. The product may or may not have two modes of operation, depending on whether a portion of the product is or is not detachable. The thermometer may comprise an infrared thermometer and may be touch-free. Any suitable connectors may be used to couple or attach one component or unit or member to another, including but not limited to bayonet locking, threaded coupling, snap-in locking, push-pull locking, latch locking, or lever locking. All electronics may be concentrated within the smart member only, or some but not all of the electronics may reside in the smart member. The product may include a processor and memory circuitry (PMC) operatively connected to a hardware-based I/O interface. The PMC may be configured to provide any processing necessary for operating the system and/or any processing shown and described herein, and may comprise a processor and a non-transitory computer-readable memory. The processor of the PMC may be configured to execute any functional modules shown and described herein in accordance with computer-readable instructions implemented on the non-transitory computer-readable memory in the PMC.

It is appreciated that certain embodiments may include a docking station or charging base which may serve as or be integrally formed with the bottom base or bottom shell or support member. The docking station may provide snap coupling (say) of the multi-functional device into the station, and may offer additional functionalities such as enhanced sound (e.g. the docking station may be integrally formed with additional built in speakers) and/or the charging base may provide mechanical stability especially, e.g., as described above, if the multi-functional smart member is spherical, in order to facilitate convenient angular orientation of the camera to ensure the child is located within the camera's field of view. The charging base may have a transparent or translucent housing for light reflection enhancement/light intensity amplification. It is appreciated that such a housing, if detachable, e.g., from a separate portable smart member, is advantageous in providing multifunctionality, because luminaire safety is enhanced by restricting the wattage of the illumination (which affects heat generation) to ensure sufficient ventilation is provided, given that higher wattage luminaires generate more heat. This in turn affects the size of the housing which is capable of ensuring sufficient ventilation is being provided, given the wattage. Thus, a safe night light with increased light intensity may be provided via one, base member of the multifunctional device, which is not intended to have grab-and-go utility, and thus may be larger, ensuring (due to the relatively large typically translucent housing) sufficient ventilation to provide sufficient wattage for the night-light, and grab-and-go utility may be provided by the other, e.g., “smart” member of the device, which may be small enough to be portable, if the smart member is not intended to provide night-light utility, hence need not have a housing large enough to ventilate the night-light. Also, the above base member may be advantageous in providing multifunctionality in the sense that the same member may serve both as a translucent cover for the night-light if provided, e.g., if the night-light illumination reflects via the base member's translucent cover and as a resonance chamber for the additional built-in speakers, if provided. Components described herein as software may, alternatively, be implemented wholly or partly in hardware and/or firmware, if desired, using conventional techniques, and vice-versa. Each module or component or processor may be centralized in a single physical location or physical device, or distributed over several physical locations or physical devices.

Included in the scope of the present disclosure, inter alia, are electromagnetic signals in accordance with the description herein. These may carry computer-readable instructions for performing any or all of the operations of any of the methods shown and described herein, in any suitable order, including simultaneous performance of suitable groups of operations, as appropriate. Included in the scope of the present disclosure, inter alia, are machine-readable instructions for performing any or all of the operations of any of the methods shown and described herein, in any suitable order.

The scope of the present invention is not limited to structures and functions specifically described herein and is also intended to include devices which have the capacity to yield a structure, or perform a function, described herein, such that even though users of the device may not use the capacity, they are, if they so desire, able to modify the device to obtain the structure or function.

Any “if-then” logic described herein is intended to include embodiments in which a processor is programmed to repeatedly determine whether condition x, which is sometimes true and sometimes false, is currently true or false, and to perform y each time x is determined to be true, thereby to yield a processor which performs y at least once, typically on an “if and only if” basis, e.g., triggered only by determinations that x is true, and never by determinations that x is false.

Features of the present invention, including operations, which are described in the context of separate embodiments, may also be provided in combination in a single embodiment. For example, a system embodiment is intended to include a corresponding process embodiment, and vice versa. Also, each system embodiment is intended to include a server-centered “view” or client centered “view”, or “view” from any other node of the system, of the entire functionality of the system, computer-readable medium, apparatus, including only those functionalities performed at that server or client or node. Features may also be combined with features known in the art, and particularly, although not limited to, those described in the Background section or in publications mentioned therein.

Conversely, features of the invention, including operations, which are described for brevity in the context of a single embodiment or in a certain order, may be provided separately or in any suitable sub-combination, including with features known in the art (particularly although not limited to those described in the Background section or in publications mentioned therein) or in a different order. “e.g.” is used herein in the sense of a specific example which is not intended to be limiting. Each method may comprise all or any subset of the operations illustrated or described, suitably ordered, e.g., as illustrated or described herein.

Devices, apparatus, or systems shown coupled in any of the drawings may in fact be integrated into a single platform in certain embodiments, or may be coupled via any appropriate wired or wireless coupling, such as but not limited to optical fiber, Ethernet, Wireless LAN, HomePNA, power line communication, cell phone, Smart Phone (e.g. iPhone), Tablet, Laptop, PDA, Blackberry GPRS, Satellite including GPS, or other mobile delivery. It is appreciated that in the description and drawings shown and described herein, functionalities described or illustrated as systems and sub-units thereof, can also be provided as methods and operations therewithin, and functionalities described or illustrated as methods and operations therewithin can also be provided as systems and sub-units thereof. The scale used to illustrate various elements in the drawings is merely exemplary and/or appropriate for clarity of presentation, and is not intended to be limiting.

1. A multi-functional device for small children, the device comprising:
a thermometer for evaluating a small child's body temperature; and, coupled thereto,
a set of functional devices including a vibrator, a speaker and/or microphone, and a night-light thereby to enable a carer or parent to efficiently and effectively minister to the small child upon her or his waking and/or when night-feeding and/or when she or he cries.

2. The device according to claim 1 and wherein the set of functional devices also comprises a baby-monitor.

3. The device according to claim 1 and also comprising a single printed circuit board, aka PCB, which controls plural devices from among the set of functional devices and the thermometer.

4. The device according to claim 3 and wherein the single printed circuit board, aka PCB, controls all devices in the set of functional devices, and also the thermometer.

5. The device according to claim 3 and also comprising a single user control device, e.g., sensor ring, which provides control, e.g., on-off control for said plural devices, e.g., for both of the night light and the speaker.

6. The device according to claim 1 and wherein said thermometer comprises a non-touch/touch-free or no-contact thermometer.

7. The device according to claim 1 wherein the night-light provides illumination and wherein the device comprises a translucent base member which is detachable from the set of functional devices and from the thermometer coupled thereto, and which is configured to engage the night-light and to receive illumination provided thereby, thereby to enhance the illumination when the night light is engaged with the base member, by virtue of the base member's translucency.

8. The device according to claim 1 and also comprising a single battery which powers plural devices from among the set of functional devices and the thermometer.

9. A child-care method comprising:
providing a multi-functional device comprising:
providing a thermometer for evaluating a small child's body temperature;
and providing, integrally formed with the thermometer, a set of functional devices including a vibrator, a speaker and/or microphone, and a night-light to enable a carer/parent to efficiently and effectively minister to the small child upon her or his waking and/or when night-feeding and/or when she or he cries.
